(19) United States
(12) Patent Application Publication
(10) Pub. No.: US 2022/0071851 A1
(43) Pub. Date: Mar. 10, 2022

(54) FLUID MANAGEMENT CONNECTION SYSTEMS AND METHODS FOR MEDICAL DEVICES

(71) Applicant: XERIDIEM MEDICAL DEVICES, INC., TUCSON, AZ (US)

(72) Inventors: PAUL WALTER MELNYCHUCK, TUCSON, AZ (US); JORDAN MCELDOWNEY, ORO VALLEY, AZ (US); CASSANDRA IVY KIMBLE, GILBERT, AZ (US); MASAOMI ENAMI, Suzuka-Shi (JP); KATE MIDEL, TUCSON, AZ (US); MIHIR MUKESHBHAI PATEL, VICTORVILLE, CA (US); CULLEN ALEXANDER TOMHAS WALSH, TUCSON, AZ (US); STRATTON ANDREW YATRON, READING, PA (US); ALYSSA JEANE BENJAMIN, TUCSON, AZ (US)

(21) Appl. No.: 17/463,659

(22) Filed: Sep. 1, 2021

Related U.S. Application Data
(60) Provisional application No. 63/074,510, filed on Sep. 4, 2020.

(51) Int. Cl. A61J 15/00; A61M 60/178; A61M 25/04; A61M 39/02; A61M 60/183; A61M 60/117; A61M 1/28; A61M 60/20; A61M 60/861; A61M 39/10

(52) U.S. Cl. CPC A61J 15/0092; A61M 2039/1033; A61M 25/04; A61M 39/0247; A61J 15/0034; A61M 60/183; A61M 60/117; A61M 1/285; A61M 60/20; A61M 60/861; A61M 39/1011; A61M 2039/0258; A61M 2039/0261; A61M 2039/027; A61M 60/178

(57) ABSTRACT

An assembly for attachment to a gastrostomy feeding tube is provided. The assembly comprises a valve connector and an anchor. The valve connector comprises a housing, a conduit traversing the housing from an inlet to an outlet; and a retaining element extending outwardly from the housing. The anchor comprises an anchor housing configured to receive a portion of the valve connector. The anchor housing has a gastrostomy feeding tube attachment feature for attaching the anchor to the feeding tube, and a valve connector retaining feature that defines a retaining space, wherein the retaining space receives the retaining element of the valve connector and is sized to enable rotation of the retaining element and the valve connector relative to the anchor such that the conduit of the valve connector maintains fluid connection with the gastrostomy feeding tube during rotation. Additional assemblies and systems are also provided for other medical devices.

### CROSS-REFERENCE TO RELATED APPLICATION(S)

This application claims priority to U.S. Provisional Application No. 63/074,510, entitled “Fluid Management Connection System for Low Profile Gastrostomy Tubes”, filed Sep. 4, 2020; the contents of which are incorporated by reference herein in its entirety.

### FIELD OF THE INVENTION

Embodiments of the present invention relate generally to medical devices and more specifically to assemblies for attachment to interventional and/or invasive medical devices, such as gastrostomy feeding tubes.

### BACKGROUND OF THE INVENTION

Various medical devices may be implanted through the skin such that a portion of the device is internal to a patient and a portion of the device is external to the patient. Such a medical device may function as an access point, allowing access for fluid, food, or other devices. In many devices, this may include a tube (e.g., catheter, wire, etc.) providing fluid communication into the patient's body, such as into a vein, their stomach, etc.

Numerous situations exist in which a body cavity needs to be catheterized through an artificial opening to achieve a medical goal. In some instances, rigid medical devices are placed through the artificial opening to aid in the procedure. In other instances, a tube is placed directly through the artificial opening. When a tube or similar is placed, the tube may limit movement of the patient, as the tube is unable to move (e.g., rotate, move through, etc.) within the artificial opening without the potential of interfering with the efficiency of the device and/or injuring/providing pain to the patient.

For example, low profile (LP) gastrostomy tubes (G-tubes) are often placed in patients who cannot ingest food or medicine orally and require an alternate route for fluid delivery. The G-tubes are inserted into an artificial opening (e.g., a surgically-created stoma), creating a direct pathway for fluids to be delivered from a feeding pump or bolus syringe into the stomach. Two primary modes of fluid delivery include continuous feeding through a feeding pump with a right-angle valve connector (designed for slow feeding), and bolus feeding through a bolus syringe with a straight valve connector (designed for fast feeding). Users can ingest fluids, such as with the continuous variation, while wearing a backpack containing a feeding pump and running the feeding tube underneath their clothing and into the G-tube.

### BRIEF SUMMARY OF THE INVENTION

As noted above, various medical devices, (e.g., gastrostomy feeding tube connection systems) are rigid at their connection point. The devices are designed to provide a direct pathway for the fluid into the desired vein or organ (e.g., the stomach). However, as presently designed, the rigidity of the connection point limits movement of the device, and in turn limits freedom for the patient.

Example embodiments of the present invention provide an improvement on interventional and/or invasive devices, wherein an interventional and/or invasive device is one which penetrates inside the body through the surface of the body with the aid or in the context of a surgical operation. Included in these devices may be rigid catheters and implanted devices connection points, that allow for fluid communication between and external source (e.g., feeding bag, battery, etc.) and an internal vein or organ (e.g., the stomach) or device (e.g. ventricular assist device).

In example embodiments, the present invention provides an assembly to increase comfort and reduce the likelihood of disconnection or malfunction of a device. The assembly may be attached to a medical device and allow rotation of the assembly in relation to the medical device to allow connected movement (e.g., rotation) about an incision site.

In some example embodiments, the assembly comprises a plurality of parts, including a valve connector and an anchor. The anchor may be secured to the medical device, such as via one or more attachment features. The valve connector may provide a fluid conduit between a tube connected to an inlet of the valve connector and an outlet into (or through) the medical device or directly into a vein or organ. In some embodiments, the assembly is formed from a thermoplastic material.

In some embodiments, the anchor is configured to provide a retaining space for the valve connector such that the valve connector may rotate freely within the retaining space. The anchor may define a retaining feature to encircle a portion of the valve connector. In this regard, the present invention provides an improvement upon current connection systems for medical devices, such as a low profile gastrostomy feeding tube connection system that, in general, allows for controlled fluid (e.g., liquid, food, or medicine) delivery to the stomach through a surgically-created stoma, bypassing part of the normal human digestive system. In some embodiments, the present invention may be used with other types of medical devices, such as with a venous access port, a ventricular assist device, or a catheter.

The system may be designed to be usable by non-medically trained users and/or caregivers in non-acute and home-care settings. In some embodiments, the present invention may be suitable for pediatric and adult use.

In an example embodiment, an assembly for attachment to a gastrostomy feeding tube may comprise a valve connector, and an anchor configured for attachment to the gastrostomy feeding tube. The valve connector may comprise a valve connector housing, wherein a conduit traverses the valve connector housing from an inlet to an outlet. The valve connector may further comprise a retaining element extending outwardly from the valve connector housing. The anchor may comprise an anchor housing configured to receive a portion of the valve connector. The anchor housing may comprise a gastrostomy feeding tube attachment feature for attaching the anchor to the gastrostomy feeding tube, and a valve connector retaining feature that defines a retaining space. The retaining space may receive the retaining element of the valve connector and be sized to enable rotation of the retaining element and the valve connector relative to the anchor. The retaining element may be sized to allow the valve connector to maintain fluid connection with the gastrostomy feeding tube during rotation.

In some embodiments, the valve connector housing may define an upper portion having an upper diameter, and a lower portion having a lower diameter which is smaller than the upper diameter. The retaining element may extend radially outward from the lower portion of the valve connector housing. In some embodiments, the valve connector retaining feature is disposed between the retaining element and the upper portion of the valve connector housing.

In some embodiments, the anchor housing may further comprise a locking piece extending about the valve connector retaining feature, and a base configured to engage with the locking piece. The engagement of the locking piece, the base, and the valve connector housing may define the retaining space. The base may further include at least one thread protruding from and extending partially about an outer circumference of the base. The locking piece may comprise at least one corresponding channel configured to receive the at least one thread. The at least one channel of the locking piece may extend at least partially about the outer circumference of an inner surface of the locking piece.

In some embodiments, the at least one thread may include a protrusion and the at least on channel may comprise a divot configured to receive the protrusion. The channel of the locking piece may angularly descend about the circumference of the locking piece.

In some embodiments, the at least one thread is a first thread and a second thread, and the at least one channel is a first channel and a second channel. The number of threads and channels may be equal. In some embodiments, the locking piece is rotatable by 90 degrees in a first direction to secure the locking piece to the base and rotatable 90 degrees in a second direction opposite the first direction to release the locking piece from securement with the base.

In some embodiments, the inlet may comprise an engagement member configured to receive a feeding tube to enable fluid communication between the feeding tube and the conduit.

In some embodiments, the valve connector is a reflux valve.

In some embodiments, the assembly is made from a thermoplastic. The thermoplastic may be polycarbonate.

In some embodiments, the valve connector may be configured to enable administration of a bolus feed. In some embodiments, the valve connector may be configured to enable administration of a continuous feed.

In another exemplary embodiment, a device enabling rotation of a supply tube of a medical device is provided. The device may include a connector, and an anchor. The connector may comprise a connector housing, wherein a conduit traverses the connector housing from an inlet to an outlet. The connector may further comprise a retaining element extending outwardly from the connector housing. The anchor may comprise an anchor housing configured to receive a portion of the connector. The anchor housing may comprise an attachment feature for attaching the anchor to the medical device, and a connector retaining feature that defines a retaining space. The retaining space may receive the retaining element of the connector and be sized to enable rotation of the retaining element and the connector relative to the anchor. The retaining element may be sized to allow the connector to maintain fluid connection with the medical device during rotation.

In some embodiments, the medical device may be an interventional or invasive device. In some embodiments, the medical device may be a gastrostomy feeding tube, a ventricular assist device, a catheter, or a venous access port.

In another example embodiment, a method of installing an assembly for a medical device is provided. The method may comprise inserting the medical device through an incision. The medical device may have a first portion internal to the incision, and a second portion external to the incision. The medical device may have an opening. An outlet of a connector may be attached to the opening of the medical device. The connector may include a connector housing, wherein a conduit traverses the connector housing from an inlet to an outlet. The connector may further comprise a retaining element extending outwardly from the connector housing. An anchor may be attached about the connector. The anchor may comprise an anchor housing configured to receive a portion of the connector. The anchor housing may comprise an attachment feature for attaching the anchor to the medical device, and a connector retaining feature that defines a retaining space. The retaining space may receive the retaining element of the connector and be sized to enable rotation of the retaining element and the connector relative to the anchor. The retaining element may be sized to allow the connector to maintain fluid connection with the medical device during rotation.

### BRIEF DESCRIPTION OF THE DRAWINGS

Having thus described the invention in general terms, reference will now be made to the accompanying drawings, which are not necessarily drawn to scale, and wherein:

FIG. 1 illustrates an example system using the assembly, in accordance with some embodiments discussed herein;

FIG. 2A illustrates a front view of an example assembly, in accordance with some embodiments discussed herein;

FIG. 2B illustrates a left side view of the example assembly shown in FIG. 2A, in accordance with some embodiments discussed herein;

FIGS. 3A-3B illustrate example exploded views of the example assembly shown in FIG. 2A, in accordance with some embodiments discussed herein;

FIG. 4 illustrates a front view of an example connector of the example assembly shown in FIG. 2A, in accordance with some embodiments discussed herein;

FIG. 5 illustrates a front perspective view of an example anchor for an example assembly, wherein a portion of locking piece of the anchor is shown in partially transparent form, in accordance with some embodiments discussed herein;

FIG. 6 illustrates a top perspective view of an example base for an anchor, in accordance with some embodiments discussed herein;

FIGS. 7A-7D illustrate various views of a cross-section of a portion of a locking piece of an anchor, in accordance with some embodiments discussed herein;

FIG. 8 illustrates a cross-sectional view of an example assembly, in accordance with some embodiments discussed herein;

FIG. 9 illustrates an example assembly attached to a gastrostomy feeding tube, in accordance with some embodiments discussed herein;

FIG. 10A illustrates another example configuration of a connector, in accordance with some embodiments discussed herein;

FIG. 10B illustrates a cross-sectional view of the connector of FIG. 10A, in accordance with some embodiments discussed herein;

FIG. 10C illustrates an example assembly attached to a gastrostomy feeding tube, where the assembly includes the example connector of FIG. 10A, in accordance with some embodiments discussed herein;

FIG. 11 illustrates an example assembly attached to a gastrostomy feeding tube, which is implanted into a patient's stomach, in accordance with some embodiments discussed herein;

FIG. 12 illustrates an example assembly for use with a venous access port, in accordance with some embodiments discussed herein;

FIG. 13 illustrates an example ventricular assist device where an example assembly such as described herein may be useful for providing increased freedom of movement for the patient, in accordance with some embodiments discussed herein;

FIG. 14 illustrates an example catheter system where an example assembly such as described herein may be useful for providing increased freedom of movement of the patient, in accordance with some embodiments discussed herein; and

FIG. 15 illustrates a flow chart of an example method of installing an example assembly, in accordance with some embodiments discussed herein.

### DETAILED DESCRIPTION OF THE INVENTION

Some example embodiments now will be described more fully hereinafter with reference to the accompanying drawings, in which some, but not all example embodiments are shown. Indeed, the examples described and pictured herein should not be construed as being limiting as to the scope, applicability or configuration of the present disclosure. Rather, these example embodiments are provided so that this disclosure will satisfy applicable legal requirements. Like reference numerals refer to like elements throughout.

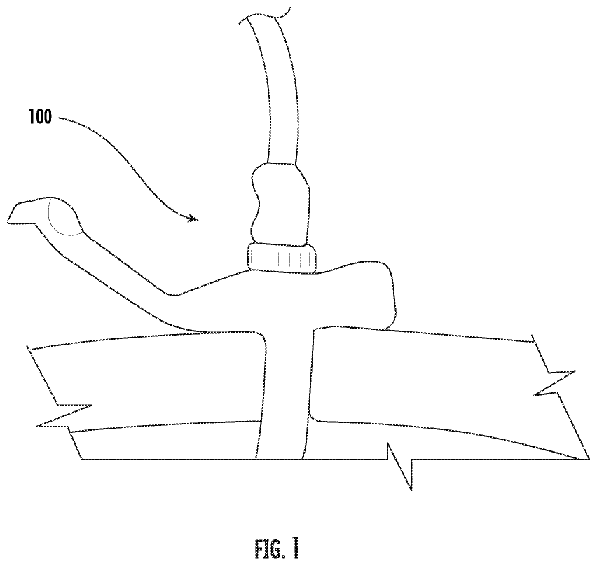

Permanent and/or semi-permanent (e.g., temporary) medical devices may be implanted through an artificial opening in the skin to allow direct access to or easier access to the implanted device. Many of these medical devices include a portion disposed internally (e.g., below the surface of the skin) and a portion disposed externally. Generally, when there is an incision site or a catheter going directly through the skin, the area around the opening may be hard to clean and manage, and further the patient may have heightened discomfort about the area as there is little to no movement (e.g., rotational or axial) allowed. In order to increase comfort and lessen the likelihood of an undesired disconnection or malfunction, an assembly may be attached to the device to allow for rotation of a tube connected to the device. FIG. 1 illustrates an example assembly 100 rotatable within a medical device when the medical device is inserted through an incision.

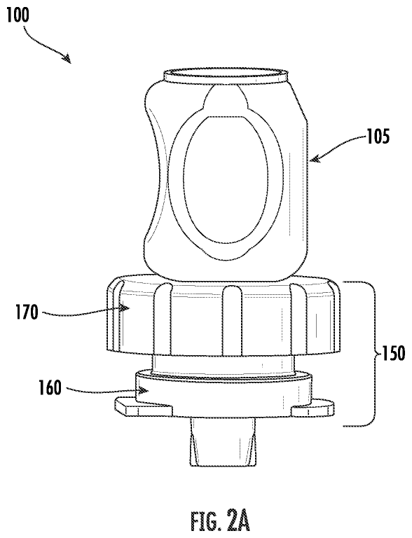

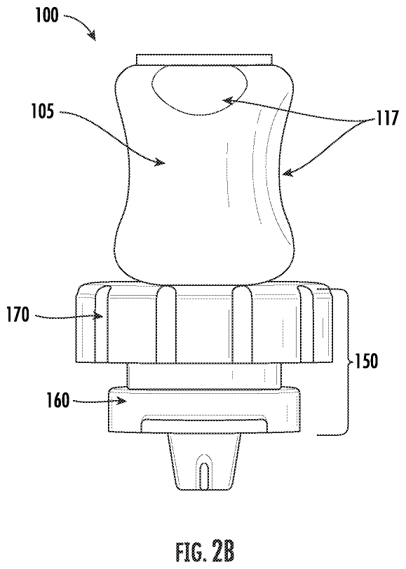

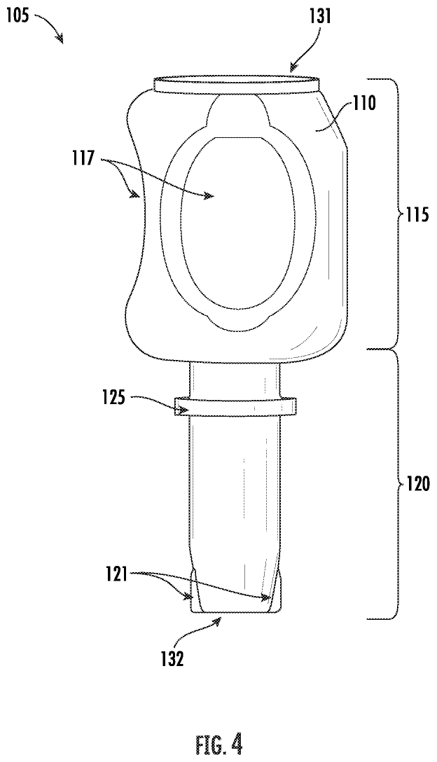

FIGS. 2A and 2B illustrate perspective views of the example assembly 100. In some embodiments, the assembly 100 may include multiple components including a connector 105 (also referred to as a valve connector) and an anchor 150. The anchor 150 may have an anchor housing 152 (shown in FIG. 5) formed of a base 160 and a locking piece 170 configured to engage with one another to form a unitary assembly (although in some embodiments, the base and the locking piece may be formed as a unitary part and may not be separate). In some embodiments, the connector 105 may extend through the anchor 150. The connector 105, as illustrated in FIG. 2B, may include one or more grip features 117, in some embodiments. The grip feature(s) 117 may be configured as a grip to aid in insertion or removal of a tube (e.g., catheter) into the connector. In some embodiments, the connector 105 includes a plurality of grip features 117, while in other embodiments there may be no grip features 117.

The assembly may be formed as a plurality of pieces, which when connected are configured to retain the connector within the anchor, such that connector is able to freely rotate within a retaining space within the anchor.

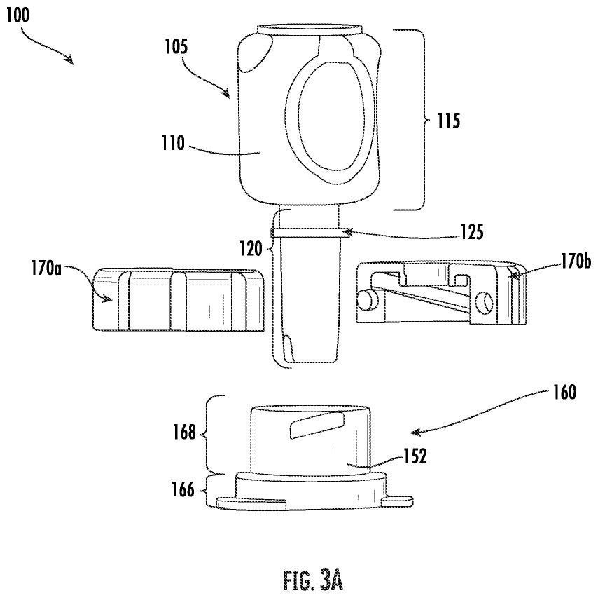

FIGS. 3A and 3B illustrate exploded views of the assembly 100. The connector 105 may include a connector housing 110 defining an upper portion 115 and a lower portion 120. The lower portion 120 may include a retaining element 125 extending radially outward about the lower portion 120.

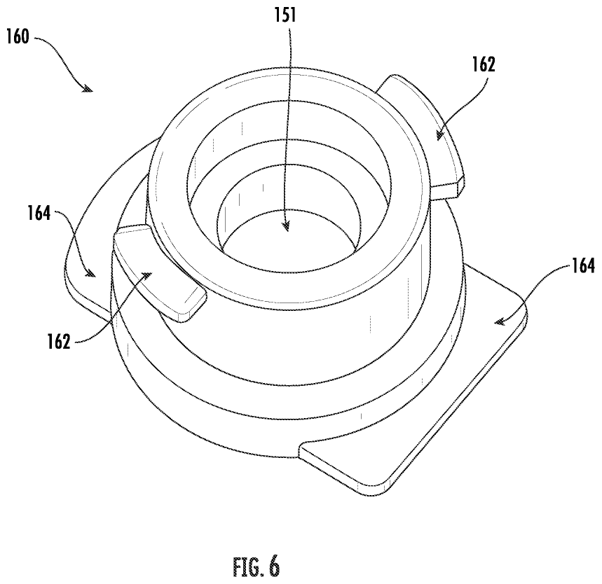

With reference to FIG. 5 (as well as FIGS. 3A and 3B), the anchor 150 may be formed as multiple pieces. In some embodiments, the locking piece 170 may be formed from pieces 170a, 170b. The portions 170a, 170b may be connected about the lower portion 120 of the connector 105. In some embodiments, each portion of the locking piece 170 may include at least one connection feature. In some embodiments, the connection feature 176 (see FIG. 7D) may be a crater and a corresponding protrusion. The crater and protrusion may form an interference fit when placed together to secure the locking piece 170. Additionally or alternatively, adhesive may be applied to the connection of the crater and protrusion to maintain secure connection between the portions 170a, 170b. In some embodiments, the connection feature 176 may be small cylindrical alignment feature, and in other embodiments the alignment feature may be circular, square, rectangular, or other shape which may have a corresponding protrusion and crater on the flat edges that align during the bonding process to ensure the locking pieces are bonded concentrically and in the same plane. In some embodiments, the connection feature 176 may be slidably engageable, snap fit or other engagement means known in the art.

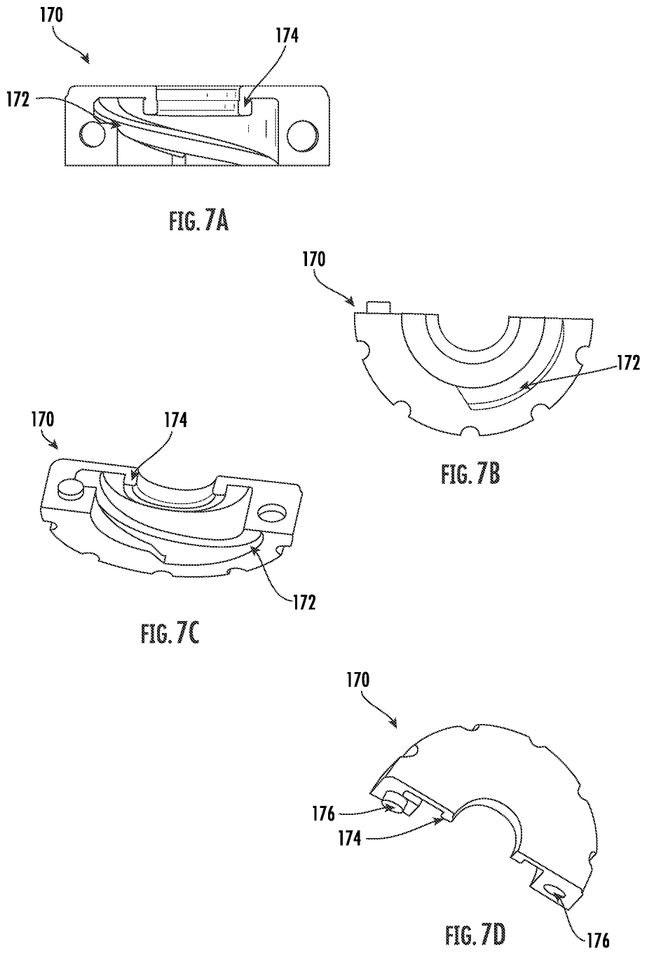

With reference to FIGS. 2A-2B, when attached with the connector 105, the locking piece 170 may be disposed about the lower portion 120 of the connector 105. A portion of the locking piece 170 may be disposed between the upper portion 115 and the retaining element 125. The locking piece 170 may engage with the base 160 to secure the connector 105 within the anchor 150. In some embodiments, the base 160 may have a varying diameter, and may define a horizontal ledge 165 (see FIG. 8). The horizontal ledge 165 may be a lower bound defining a retaining space 158 within the anchor 150 to receive the retaining element. The retaining space 158 may be bound on the top side by a retaining feature 174 of the locking piece 170, and the retaining feature 174 may be disposed between an upper portion of the retaining element and the upper portion 115 of the housing 110. Notably, as detailed herein, the retaining space 158 is designed with additional space relative to the size of the retaining element 125 of the connector 105 in order to enable movement (e.g., rotational and/or vertical) of the retaining element 125 therein.

With reference to FIGS. 3A-3B, the base 160 may define a top section 168 and a lower section 166. In some embodiments, when assembled, the top section 168 may be outside of a medical device or an incision point, and the bottom section 166 may be within the device or incision point (e.g., internal to the patient).

The assembly components may be formed from a thermoplastic material. For example, the connector housing and the anchor housing may be formed from polylactic acid, polycarbonate, polyethylene, polypropylene or a combination thereof. The components may be printed, molded, or formed in another manner.

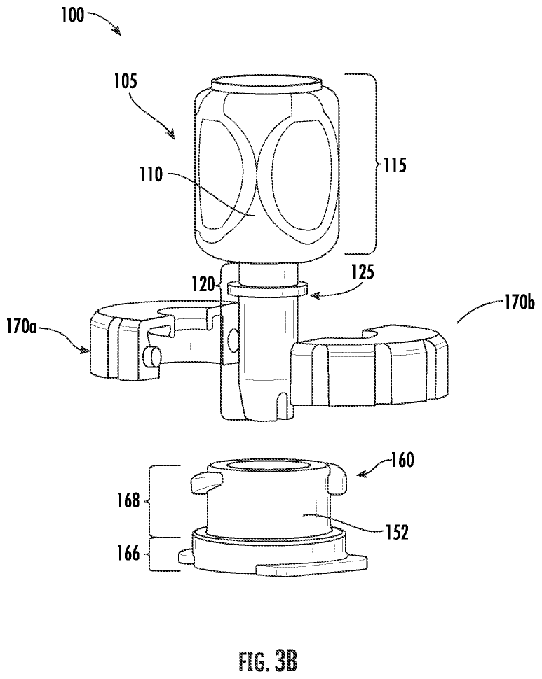

The connector may provide for fluid communication from a tube or other supply feed to a medical device or vein/organ, through a conduit within the connector housing. FIG. 4 illustrates an example connector 105 comprising a connector housing 110 defining an upper portion 115 and a lower portion 120.

The connector housing 110 may define a conduit 130 (see. e.g., FIG. 8) traversing from an inlet 131 within the upper portion 115 to an outlet 132 in the lower portion 120. In some embodiments, the conduit may have a constant diameter, while in other embodiments, the conduit may have a changing diameter, or multiple diameters along the length of the conduit. Varying conduit diameters may allow the connector 105 to be utilized with multiple or different sizing of external and internal tubes and/or catheters (e.g. 12- and 16-French size variations).

In some embodiments, the upper portion 115 may be configured as a bulbous shape, allowing for increased ergonomic features. For example, in some embodiments, the upper portion 115 may include a grip feature 117 disposed about the exterior of the connector housing 110. The grip feature 117 may provide a grip for inserting and removing tubes into the inlet 131. In some embodiments, the conduit 130 may include an internal mechanism for connection between the inlet 131 and a fluid tube, for example a feeding tube (e.g., threading, snap features, etc.).

With reference to FIG. 8, the upper portion 115 may have an upper diameter D_{U} which may be constant along the height, while in some embodiments, the upper diameter D_{U} may change along the height. The upper diameter D_{U} may vary based on the cross-section of the upper portion 115. For example, in some embodiments, the upper portion 115 may not be symmetrical and thus have a changing diameter. Although, “diameter” is used herein it should be understood that “diameter” is used to refer to any length between two points on opposite sides of a housing.

The lower portion 120 may descend below the upper portion 115 to be received by the anchor. In some embodiments, the lower portion 120 may be configured as a cylinder having a lower diameter D_{L}, wherein the lower diameter D_{L} is smaller than the upper diameter UD. The lower portion 120 may include a retaining element 125 (although in some embodiments, the retaining element may be positioned elsewhere on the housing 110).

The retaining element 125 may extend radially outward from the housing of the lower portion 120. The retaining element 125 may define a retaining element diameter D_{RE}. The retaining element diameter D_{RE} may be larger than the lower diameter D_{L}, and may be smaller than the upper diameter D_{U}. In some embodiments, the retaining element 125 may extend continuously about the lower portion 120, while in other embodiments, the retaining element 125 may be formed as a one or more discontinuous radially outwardly extending protrusions. The radially extending protrusions may be spaced along the circumference of the lower portion 120.

In some embodiments, the diameter of the lower portion D_{L} may decrease at a bottom region of the lower portion near the outlet 132. In some embodiments, the bottom region of the lower portion includes at least one connection mechanism 121 to aid connection between the lower portion and a tube, or other device. In some embodiments, the connection mechanism may be disposed on the external surface of the housing, while in other embodiments the connection mechanism may be disposed on an interior surface about the outlet of the conduit 130. In some embodiments, the connection mechanism 121 may be configured for an interference fit, a snap fit, as a threaded engagement, or other engagement means.

As noted herein, the anchor housing is configured to provide a retaining space for the connector such that the connector may rotate freely within the retaining space. Additionally, the anchor housing is removably fixed, to or within a medical device, (e.g., G-tube) to secure the connector to the medical device to allow fluid communication through the conduit 130 from the inlet 131 to the outlet 132, and into the medical device and/or vein/organ.

FIG. 5 illustrates an example anchor 150. As illustrated, a portion of the locking piece 170 is partially transparent, thereby illustrating the connection feature between the locking piece 170 and the base 160. The anchor 150 comprises an anchor housing 152 wherein the anchor housing defines a locking piece 170 having a retaining feature 174, and a base 160 having an attachment feature 164.

In some embodiments, the attachment feature may be configured as a pair of planar extensions diametrically opposite one another about the bottom section 166. The attachment feature 164 may secure the anchor 150 within a medical device or an incision site. In other embodiments, the attachment feature may be configured as at least one thread about an interior surface of the bottom section 166. The at least one thread may be configured to engage with a corresponding thread about an exterior surface of a neck of a medical device. In some embodiments, the threads may be configured to engage by twisting, and/or applying force.

The anchor housing 152 may include a retaining feature 174. The retaining feature 174 may be formed integral to the locking piece 170, such that the locking piece 170 engages with the base 160, and the valve connector housing, to define a retaining space. The retaining space 158 (shown in FIG. 8) is configured to receive the retaining element 125 of the connector housing 110. The retaining feature 174 and the base 160 may be sized to enable rotation of the connector 105 within the retaining space 158 relative to the anchor 150 while still preventing removal of the retaining element 125 from the retaining space 158 (and, thus, preventing removal or disengagement of the connector from the medical device or incision site). The anchor housing 152 may define an opening 151 traversing the anchor 150. The opening 151 may be sized to receive and retain the lower portion 120 of the connector 105.

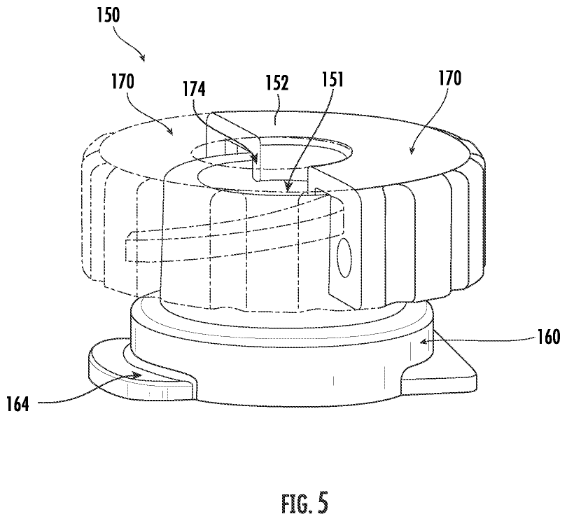

FIG. 6 illustrates a perspective view of the base 160 of the anchor housing. The base 160 may include at least one thread 162 on an exterior surface of the top section 168. The at least one thread 162 may extend along the surface about a portion of the exterior circumference of the top section 168. In some embodiments, the thread 162 may extend along half of the circumference, a third of the circumference, a quarter of the circumference, or other designed portion. In some embodiments, the base 160 may include one thread 162, while in other embodiments, the base may include a plurality of threads 162 spaced about the exterior surface of the top section 168. The at least one thread 162 may be level across the thread, while in other embodiments, the thread may angularly descend along the exterior surface.

In some embodiments, the thread may be configured to be received by a corresponding channel within the locking piece. The thread 162 and channel 172 may have corresponding shapes, slopes, thickness, and other characteristics. In some embodiments, the thread 162 may include a divot or protrusion to interlock with a receiving segment within the channel of the locking piece. In some embodiments, the thread 162 may include a receiving segment, while the channel 172 may include a divot or protrusion to interact with the receiving segment. In some embodiments, when the thread 162 and the channel 172 are engaged, the locking piece 170 is locked onto the base 160 until acted upon by a greater force in the opposite direction.

With reference to FIG. 8, in some embodiments, base 160 may be configured to have a varying inner diameter. The base 160 may have a first diameter D₁ within the top section, wherein the first diameter D₁ slightly larger than the retaining element diameter D_{RE} such that the retaining element may rotate within the portion of the base 160 having the first diameter D₁. In some embodiments, the retaining space 158 is partially defined by the portion of the base having the first diameter D₁.

In some embodiments, the base may have a second diameter D₂. The portion of the base 160 having the second diameter D₂ may be the below and adjacent to the portion of the base having the first diameter D₁. The second diameter D₂ may be smaller than both the first diameter D₁ and the retaining element diameter D_{RE} and be larger than the lower portion diameter D_{L}. In such embodiments, a horizontal or substantially horizontal ledge 165 is formed at the transition point between the first diameter D₁ and the second diameter D₂. Therein the retaining space 158 is partially defined within the portion of the base having the first diameter D₁ between the horizontal plane and the retaining feature 174.

The base 160 may further define at least one attachment feature 164 on the bottom portion 166. As discussed above, the attachment feature 164 may be configured to connect the base 160 to a medical device so as to secure the assembly within the medical device. For example, the attachment feature 164 may be configured to be inserted into a gastrostomy tube (G-tube). To insert the base into the G-tube, the base may be tilted to be inserted within an opening of the tube. After the base is inserted into the medical device the connector may be inserted through the opening 151, and the locking piece may be secured about the lower portion 120 of the connector 105.

FIGS. 7A-7D illustrate various views of a cross section of the locking piece 170. As illustrated in FIG. 7A, the locking piece 170 may include a channel 172 to receive a thread 162 of the base. In some embodiments, the channel may be parallel to a top surface of the locking piece, and in other embodiments the channel may angularly descend about the circumference of the interior surface. In some embodiments, the channel may include at least one cut out, or protrusion along the channel to receive a protrusion, or interact with a cut out of the thread. The anchor is secured by the interaction of the channel 172 receiving the thread 162, such that the locking piece cannot be removed by an indirect application of force.

In some embodiments, the locking piece 170 may have a plurality of channels 172 within the inner wall, and the number of channels 172 may correspond in number, and configuration (e.g., slope, thickness, shape) to the threads 162 on the base 160. In some embodiments, the channel 172 may have a longer length than the thread 162 such that the thread 162 may be received and secured within the channel 172.

Referring back to FIG. 5, as illustrated, the channel 172 and the thread 162 may engage to form the anchor 150. The locking piece 170 may be configured to engage the thread 162 and rotate the thread 162 through the channel. In some embodiments, the locking device may be rotated up to 90 degrees into the locked position while in other embodiments the anchor may be in the locked position after the locking piece 170 is rotated up to 70 degrees, up to 50 degrees, up to 30 degrees, or up to 15 degrees.

With the anchor 150 attached to the connector 105, the retaining feature 174 may be configured to at least partially block (e.g., encircle) the lower portion 120 of the connector 105. The retaining feature 174 may define an upper inlet of the opening 151 within the anchor. The retaining feature 174 may define an opening diameter D_{O}. The opening diameter D_{O} may be larger than the lower diameter D_{L} and smaller than both the diameter of the retaining element D_{RE} and the upper portion D_{U}. In some embodiments, the opening diameter D_{O} may be essentially equivalent to the second diameter D₂ within the base 160. The opening diameter D_{O} may be sized to enable rotation of the lower portion 120 within the opening 151.

The retaining feature 174 may have a height which extends from an upper surface of the retaining element 125 to approximately the upper portion 115 of the connector housing 110. In this regard, the retaining feature 174 may axially fit between the upper portion 115 of the housing and the retaining element 125. In some embodiments, the height of the retaining feature 174 may be sized such that the valve connector 105 may rotate and have minimal axial movement. Notably, the positioning and the configuration of the locking piece 170 engaged with the base 160 may yield a stationary anchor 150 while allowing rotation of the connector 105, and other elements (e.g., a feeding tube) that are connected to the connector 105.

The locking piece 170 may be configured to engage an inner surface and an exterior surface of the base 160. In some embodiments, the retaining feature 174 may descend adjacent to and interior of an interior surface of the base, and an inner surface of the locking piece may surround at least a portion of an exterior surface of the base 160. The engagement of the locking piece 170 and the base 160 define the anchor 150 which may receive and retain the connector 105 within the retaining space 158.

FIG. 8 illustrates a cross-sectional view of the assembly. As discussed above the diameters of various components are sized to enable the connector 105 to freely rotate, 360 degrees, within the retaining space 158 as defined by the engagement of the base 160, and the retaining feature 174. The retaining space 158 provides rotational movement (and may provide some limited vertical movement, without allowing removal of the connector 105 from the anchor 150.

In some embodiments, a higher friction force may be designed between the base 160 and locking piece 170 than between the valve connector 105 and locking piece 170 to enable the system 100 to stay in place (e.g., on the medical device (e.g., G-Tube)) when the valve connector 105 is rotated.

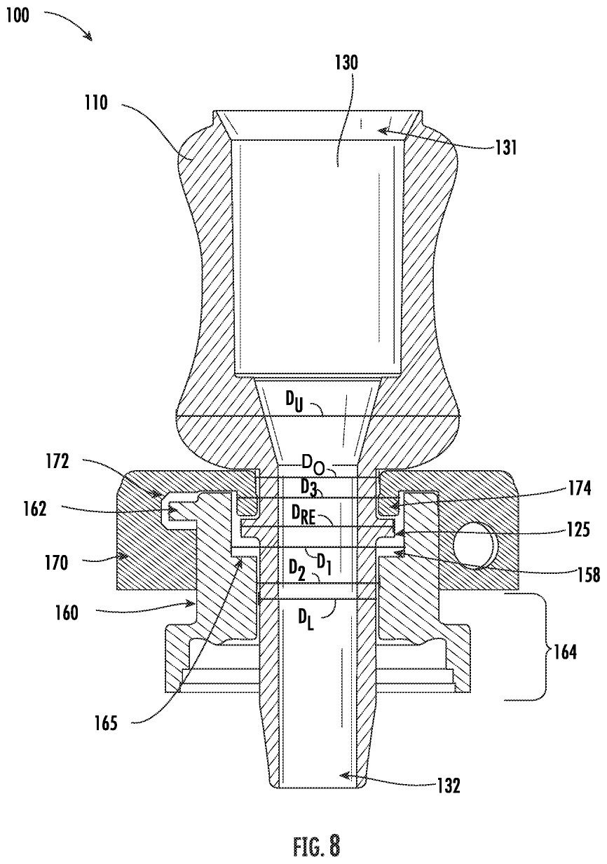

The assembly may be used in a bolus feed configuration that provides a feeding tube in a straight, or nearly straight, direction into the assembly. FIG. 9 illustrates an example assembly 200 within G-Tube 280 having a bolus feed configuration. A bolus feeding configuration may be beneficial when fast feeding is desired. The base 260 may be partially disposed within the G-Tube 280. For example, the bottom section may be positioned completely within the G-tube 280, and the top section may be positioned partially within the G-tube or completely out of the G-tube 280. The locking piece 270 may be fastened about the top portion of the base 260 and the connector 205 may be secured within the anchor. A feeding tube 282 may be connected to the connector 205 such that the feeding tube 282 is in fluid communication with the G-tube, and may supply a fluid (e.g., nutrient dense feed) from the feeding tube 282, through the conduit 230, and to the desired destination (e.g., the stomach). The present embodiment allows rotation of the feeding tube 282 and the valve connector 205 relative to the G-Tube 280. In the illustrated embodiment, the valve connector 205 is a reflux valve configured to allow fluid into the valve, while preventing fluid (e.g., stomach acid) from flowing out of the valve.

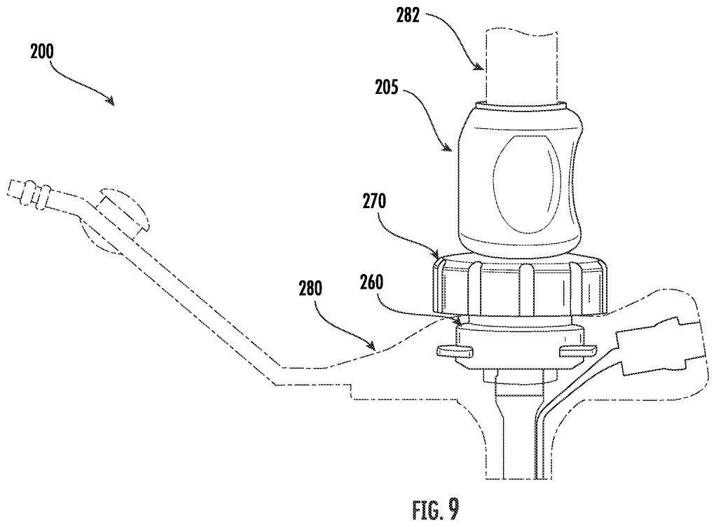

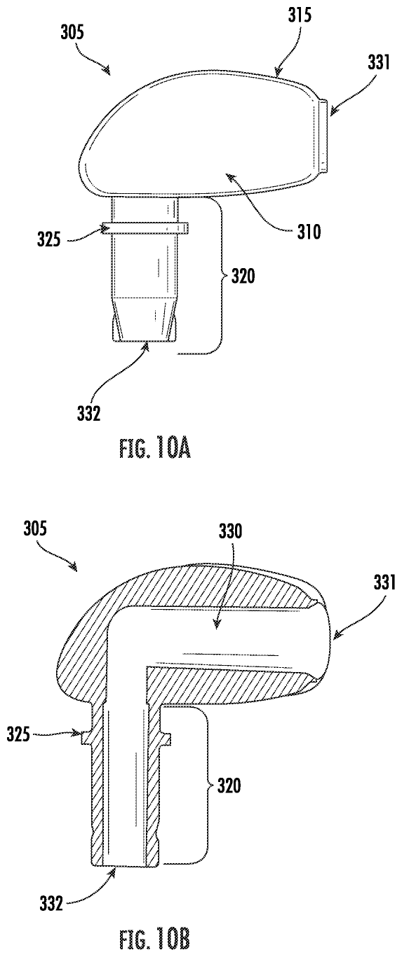

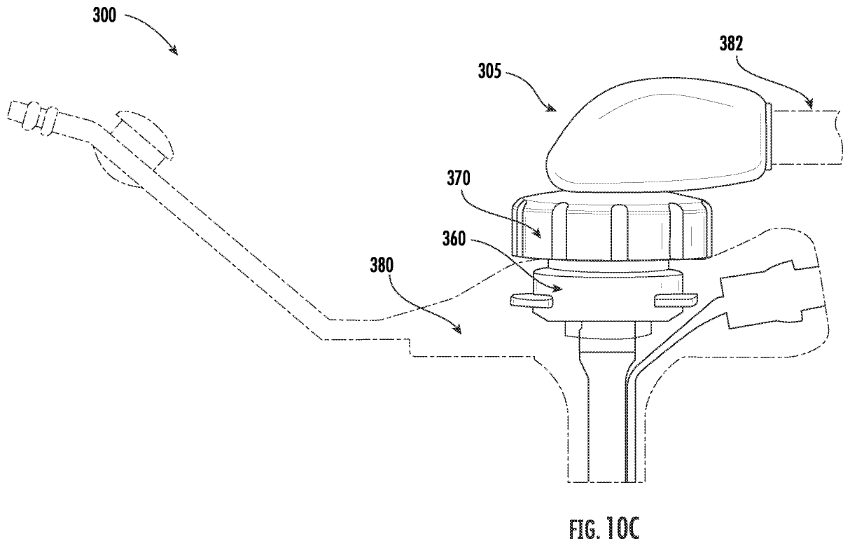

Various embodiments may require varying levels of fluid communication between the inlet and the outlet of the connector. In some embodiments, a slower, steady fluid delivery may be desired. FIG. 10A-10B illustrate an example configuration of a connector 305 for use within an assembly. The connector 305 may be similar to the connector 105, illustrated in FIG. 4. The connector 305 may include a housing 310 defining an upper portion 315 and a lower portion 320. The upper portion 315 may include an inlet 331, and the lower portion 320 may include an outlet 332. In some embodiments, the housing may define a conduit 330 traversing the connector 305 from the inlet 331 to the outlet 332. In some embodiments, the conduit 330 may include an angle within the upper portion 315. The angle of the conduit may change the flow rate of a fluid traversing the conduit 330. In some embodiments, the angle may be up to 90 degrees, 80 degrees, 70 degrees, or other angle, as measured from vertical, so as to decrease or change the flow rate. In some embodiments, the conduit may have a constant diameter, while in other embodiments, the conduit may have a changing diameter. A changing diameter may allow the connector 305 to be utilized with multiple or different sizing of external tubes. In some embodiments, the upper portion 315 may be configured to receive a tube, for example a feeding tube. The lower portion 320 may be substantially similar to the lower portion 120 of the straight configuration of the valve connector.

FIG. 10C illustrates an example of a continuous feed assembly. The assembly 300 is disposed within G-Tube 380 and is attached, via the angled valve connector 305 to a continuous feeding tube 382. A continuous feeding configuration may be beneficial for slow continuous fluid communication between the tube 382 and the G-tube 380. Similar to the example assembly 200, illustrated in FIG. 9, the base 360 may be partially disposed within the G-tube 380 such that the bottom portion is completely within the G-tube 380, and the top portion may be partially within the G-tube, or completely out of the G-tube 380. The locking piece 370 may be fastened about the top portion of the base 360 and the connector 305 may be secured within the anchor 360. The tube 382 may be connected to the connector 305 such that the tube 382 is in fluid communication with the G-tube, and may supply a fluid from the tube 382 through the connector conduit, to the desired destination (e.g., the stomach). Notably, the illustrated embodiment provides for rotation of the connector 305 within the anchor, while maintaining the connection.

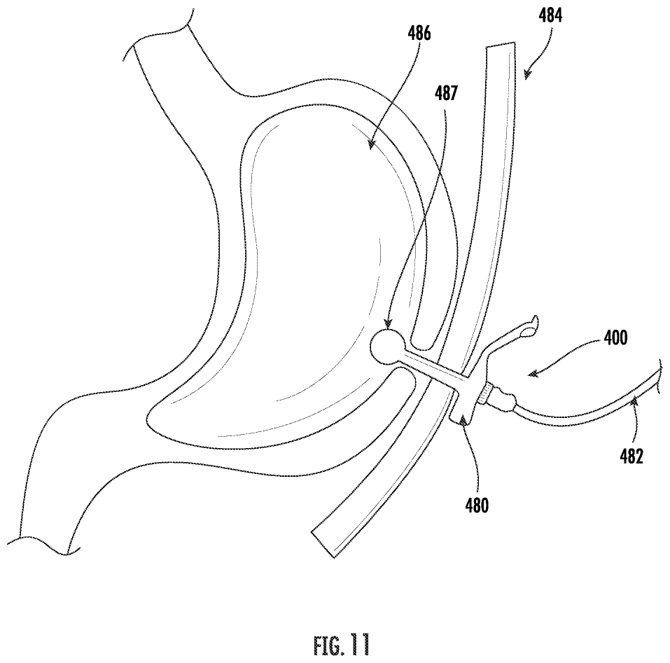

FIG. 11 illustrates an example assembly inserted into a stomach. The G-tube 480 is inserted into the stomach 486 though an incision though the skin 484. The G-tube 480 may be held into place by an inflated silicon balloon 487. The assembly 400 (with either the straight, or angled configuration of the connector) may be inserted and secured within the G-tube 480. In the illustrated embodiment, a feeding tube 482 may be connected into the assembly to provide a feeding solution through the assembly into the stomach. The locking piece and the anchor allow the fluid to flow into the stomach, without leakage between the interior and exterior parts. In some embodiments, the assembly may be provided with a reflux valve such that the stomach contents may not flow into the G-tube and assembly. In some embodiments, the tube may be a feeding tube 482 being a common 12- or 16-gauge French line.

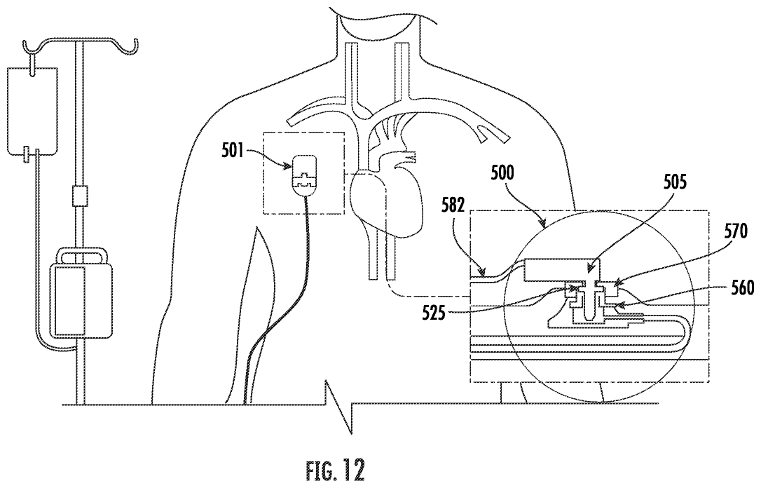

The assembly may be used in conjunction with various medical devices to provide maneuverability and a safeguard between the external connection lines and the interior medical devices and/or other tubes. FIGS. 12-14 illustrate various example embodiments for use in other medical devices (besides a G-Tube), although any medical device is contemplated.

FIG. 12 illustrates an example assembly 500 for use with a venous access port 501. A venous access port may be inserted under the skin and be semi-permanent (e.g., inserted for weeks or months). A venous access port is a device implanted under the skin including a reservoir attached to a vein, to aid in supplying or removing fluid from the vein (e.g., intravenous (IV) treatments, blood draws, etc.). In use, a needle or supply device may be inserted through the skin into the reservoir for a period of time to supply a fluid to a vein and/or take fluid therefrom (e.g., blood draw). In some embodiments, the needle may not be moveable, (e.g., rotatably or axially) which may lead to discomfort and/or lack of movement while the port is connected to the supply device (e.g., IV). An assembly 500 may be fixed to and implanted with the port to allow rotation of the supply line. In some embodiments, a base 560 may be fixed to the reservoir as discussed above. The base 560 may traverse the incision site 590 such that a portion of the base is internal and a portion of the base is external to the incision site. In some embodiments, the skin may heal around the incision site and the base.

A valve connector 505 may be disposed through the base 560 to provide fluid communication through a conduit connecting the supply line 582 to the reservoir. A locking piece 570 may be placed about the valve connector 505, securing the retaining element 525 within the base 560 and locking piece 570 such that the valve connector 505 is configured to rotate within the base 560. The locking piece 570 is connected to the external portion of the base 560 (e.g., rotating the thread within the channel) thereby securing the valve connector within the base. In some embodiments, a closure (e.g., cap or cover) may be provided on the base 560 and/or inlet of the valve connector 505 to close and secure the base 560 or valve connector 505 in between uses or attachment to an exterior tube 582. The assembly 500 may afford comfort and peace of mind that minimal movements may displace the catheter, and result in rehabilitative surgery or reinsertion of the device, or issues and/or delays with treatment, or procedures. Although, the port is shown in the chest, the port, and assembly 500 may be implanted any acceptable area (e.g., the arm, and abdomen).

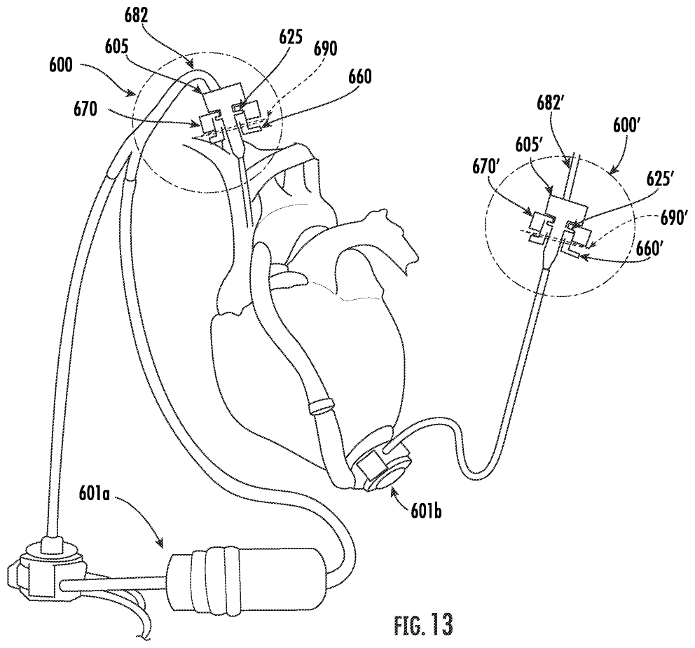

FIG. 13 illustrates two example assemblies 600, 600′ for use within a ventricular assist device. A ventricular assist device (VAD) is used when a patient has heart failure. Depending on the type and severity of the failure a right ventricular assist device (RVAD) 601a, a left ventricular assist device (LVAD) 601b, or a biventricular assist device (BIVAD) may be utilized. Each of the devices may either be paracorporeal or implantable. A paracorporeal VAD utilizes an external pump which takes blood out of the body, passes through the pump, and returns into and through the body, and an implantable VAD utilizes a pump implanted within the body. Both systems use an external (e.g., outside of the body) energy source.

An example embodiment of a paracorporeal VAD is illustrated in FIG. 13. In some embodiments, a tube (e.g., long thin catheter) is inserted into the femoral vein or artery. The catheter may be inserted at an incision point and may be required to be stationary, thereby inducing discomfort and restricting movements. An assembly 600 may be implanted with the catheter to allow rotation of the supply line between the pump and the artery. In some embodiments, an incision site 690 may be made where the catheter is to be inserted. The base 660 may be partially disposed beneath the incision site 690. A connector 605 may be disposed within the base 660 and a locking piece 670 may be disposed and attached to the base as described above. The catheter may be inserted thought the assembly 600 such that the catheter 682 may be able to rotate within the base 660.

An example embodiment of an implanted VAD is further illustrated in FIG. 13. An implanted VAD may be used with an external battery to supply power to an internal pump which moves blood through the pump, thereby bypassing the heart chambers. A drive line stems from an external control site, to enter the body and provide electricity to the pump to maintain the flow of blood through the VAD.

The assembly 600′ may be inserted through an incision site 690′. A base 660′ may be implanted within the incision site 690′ to secure the connector 605′ within the incision. A locking piece 670′ may be disposed about the connector 605′ such that the retaining element 625′ of the connector 605′ is secured between, and freely rotatable within the locking piece 670′ and the base 660′. In some embodiments, a first tube 682′ is connected to the inlet of the connector 605′, and a second tube is connected to the outlet of the connector 605′. The drive line may traverse from a power supply, through the first tube 682′, the connector 605′ and the second tube to reach the internal pump. Installing the assembly 600′ about the incision site may increase patient comfort while maintaining the reliability of the VAD.

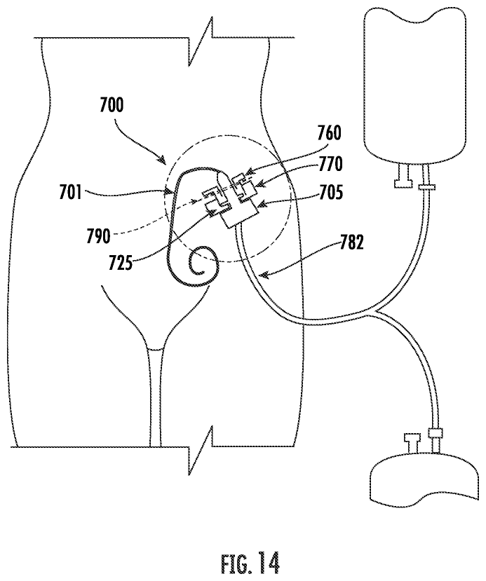

FIG. 14 illustrates an example assembly 700 for use within a catheter 701. FIG. 14 illustrates a peritoneal dialysis catheter (PDC), but any appropriate catheter may be utilized. A PDC may be utilized when the kidneys are not adequately removing waste products from the blood. A catheter is inserted in the abdomen into the peritoneum. When inserted, the catheter may create discomfort about the incision site, and once healed, the area around the incision may be tender, and the catheter may be stationary (e.g., no movement, axially or rotationally). To alleviate the discomfort, in some embodiments, an assembly 700 may be placed at the incision site 790 in the abdomen to allow movement of the tubes 782 and reduce the irritation from moving. A base 760 may be inserted through the incision site 790 to receive a connector 705. A locking piece 770 may be disposed about the connector 705 such that the retaining element 725 of the connector 705 is secured between, and freely rotatable within the locking piece 770 and the base 760. Although a peritoneal dialysis catheter is discussed, the assembly may be used with any acceptable catheter.

### Example Flowchart(s) and Operations

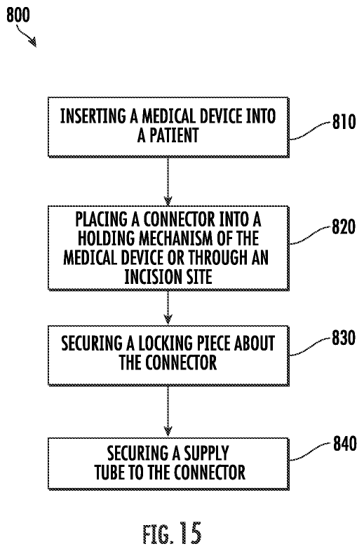

Some embodiments of the present invention provide methods, apparatus, and computer program products related to the presentation of information according to various embodiments described wherein. Various examples of the operations performed in accordance with embodiments of the present invention will now be provided with reference to FIG. 15. Notably, various operations may be performed with various example embodiments described herein.

FIG. 15 illustrates a flow chart according to an example method 800 of inserting and using an assembly such as described herein. The method of using the assembly depicted in FIG. 15 may include inserting a medical device into a patient at operation 810. The medical device may be an interventional and/or invasive device. Such devices may include, but is not limited to, a G-tube, a venous access port, a ventricular assist device, a catheter, a peritoneal dialysis catheter, a peripherally inserted central catheter (PICC) line, a central line, a nephrostomy tube, wound drains, peripheral vascular access device, central venous access device, insulin pump, an infusion pump, and cochlear implants. The method 800 may continue by placing a base into a medical device or through an incision site, and placing a connector into the base at operation 820. In some embodiments, the base may be placed within a medical device, while in other embodiments the base may be placed directly in the incision. The method 800 may continue by securing a locking piece to the base, such as about a lower portion of the connector, at operation 830. In some embodiments, a supply tube is connected to the connector at operation 840. Accordingly, rotational movement of the supply tube and connector relative to the anchor, medical device, and/or incision site is achieved without causing patient discomfort or other complications.

### CONCLUSION

Many modification and other embodiments of the inventions set forth herein will come to mind to one skilled in the art to which these inventions pertain having the benefit of the teaching presented in the foregoing description and the associated drawings. Therefore, it is to be understood that the embodiments of the invention are not to be limited to the specific embodiments disclosed and that the modification and other embodiments are intended to be included within the scope of the invention. Moreover, although the foregoing descriptions and the associated drawings describe example embodiments in the context of certain example combinations of elements and/or functions may be provided by alternative embodiments without departing from the scope of invention. In this regard, for example, different combinations of elements and/or functions than those explicitly described above are also contemplated within the scope of the invention. Although specific terms are employed herein, they are used in a generic and descriptive sense only and not for purposes of limitation.

That which is claimed:

1. An assembly for attachment to a gastrostomy feeding tube, the assembly comprising:
a valve connector comprising:
a valve connector housing;
a conduit traversing the valve connector housing from an inlet to an outlet; and
a retaining element extending outwardly from the valve connector housing; and
an anchor configured for attachment to the gastrostomy feeding tube, the anchor comprising:
an anchor housing configured to receive a portion of the valve connector, the anchor housing comprising:
a gastrostomy feeding tube attachment feature for attaching the anchor to the gastrostomy feeding tube; and
a valve connector retaining feature that defines a retaining space, wherein the retaining space receives the retaining element of the valve connector and is sized to enable rotation of the retaining element and the valve connector relative to the anchor such that the conduit of the valve connector maintains fluid connection with the gastrostomy feeding tube during rotation.

2. The assembly of claim 1, wherein the valve connector housing further defines an upper portion having an upper diameter, and a lower portion having a lower diameter, wherein the upper diameter is larger than the lower diameter.

3. The assembly of claim 2, wherein the retaining element extends radially outward from the lower portion of the valve connector housing.

4. The assembly of claim 2, wherein the valve connector retaining feature is disposed between the retaining element and the upper portion of the valve connector housing.

5. The assembly of claim 1, wherein the anchor housing further comprises:
a locking piece extending about the valve connector retaining feature; and
a base configured to engage with the locking piece, such that engagement of the locking piece and the base encloses, along with the valve connector housing, the retaining space.

6. The assembly of claim 5, wherein the base comprises at least one thread protruding from and extending partially about an outer circumference of the base; and
wherein the locking piece comprises at least one channel configured to receive the at least one thread, wherein the at least one channel extends at least partially about the outer circumference of an inner surface of the locking piece.

7. The assembly of claim 6, wherein the at least one thread comprises a protrusion and the at least one channel comprises a divot to receive the protrusion.

8. The assembly of claim 6, wherein the at least one channel angularly descends about the circumference of the locking piece.

9. The assembly of claim 6, wherein the at least one thread is a first thread and a second thread, wherein the second thread is disposed opposite the first thread; and
wherein the at least one channel is a first channel and a second channel, wherein the second channel is disposed opposite the first channel.

10. The assembly of claim 6, wherein the locking piece is rotatable by 90 degrees in a first direction to secure the locking piece to the base, and rotatable 90 degrees in a second direction opposite the first direction to release the locking piece from securement with the base.

11. The assembly of claim 1, wherein the inlet comprises an engagement member configured to receive a feeding tube to enable fluid communication between the feeding tube and the conduit.

12. The assembly of claim 1, wherein the valve connector is 360 degree rotatable within the anchor.

13. The assembly of claim 1, wherein the valve connector is a reflux valve.

14. The assembly of claim 1, wherein the assembly is made from a thermoplastic.

15. The assembly of claim 1, wherein the assembly is made from polycarbonate.

16. The assembly of claim 1, wherein the valve connector is configured to enable administration of a bolus feed.

17. The assembly of claim 1, wherein the valve connector is configured to enable administration of a continuous feed.

18. A device enabling rotation of a supply tube of a medical device, the device comprising:
a connector comprising:
a connector housing;
a conduit traversing the connector housing from an inlet to an outlet; and
a retaining element extending outwardly from the connector housing; and
an anchor configured for attachment to the medical device, the anchor comprising:
an anchor housing configured to receive a portion of the connector housing, the anchor housing comprising:
an attachment feature for attaching the anchor to the medical device; and
a connector retaining feature that defines a retaining space, wherein the retaining space receives the retaining element of the connector and is sized to enable rotation of the retaining element and the connector relative to the anchor such that the conduit of the connector maintains fluid connection with the medical device during rotation.

19. The device of claim 18, wherein the medical device is an interventional or invasive device.

20. The device of claim 18, wherein the medical device is a gastrostomy feeding tube.

21. The device of claim 18, wherein the medical device is a ventricular assist device.

22. The device of claim 18, wherein the medical device is a catheter.

23. The device of claim 18, wherein the medical device is a venous access port.

24. A method of installing an assembly for a medical device, the method comprising:
inserting the medical device through an incision, wherein a first portion of the medical device is on an internal side of the incision and a second portion of the medical device is on an external side of the incision, wherein the medical device comprises an opening;
attaching an outlet of a connector to the opening of the medical device, the connector comprising:
a connector housing;
a conduit traversing the connector housing from an inlet to an outlet; and
a retaining element extending outwardly from the connector housing; and
attaching an anchor to the medical device about the connector, wherein the anchor comprises:
an anchor housing configured to receive a portion of the connector housing, the anchor housing comprising:
an attachment feature for attaching the anchor to the medical device; and
a connector retaining feature that defines a retaining space, wherein the retaining space receives the retaining element of the connector and is sized to enable rotation of the retaining element and the connector relative to the anchor such that the conduit of the connector maintains fluid connection with the medical device during rotation.
